(19) United States
(12) Patent Application Publication
(10) Pub. No.: US 2025/0197334 A1
(43) Pub. Date: Jun. 19, 2025

(54) PROCESS FOR REACTING OLEFINS WITH PRIOR SEPARATION OF THE FEED STREAM

(71) Applicant: Evonik Oxeno GmbH & Co. KG, Marl (DE)

(72) Inventors: Alexander Brächer, Hackenheim (DE); Robert Franke, Marl (DE); Anna Chiara Sale, Duesseldorf (DE); Dirk Fridag, Haltern am See (DE); Ana Markovic, Haltern am See (DE); Johannes Knossalla, Everswinkel (DE); Peter Kucmierczyk, Herne (DE)

(73) Assignee: Evonik Oxeno GmbH & Co. KG, Marl (DE)

(21) Appl. No.: 18/975,354

(22) Filed: Dec. 10, 2024

(30) Foreign Application Priority Data
Dec. 18, 2023 (EP) 23217518.2

(51) Int. Cl. C07C 45/50; C07C 5/03; C07C 7/04

(52) U.S. Cl. CPC C07C 45/505; C07C 5/03; C07C 7/04; C07C 2521/04; C07C 2521/06; C07C 2521/08; C07C 2521/10; C07C 2523/42; C07C 2523/44; C07C 2523/46; C07C 2523/755

(57) ABSTRACT

A process reacts olefins having 4 to 20 carbon atoms and the feed stream used, containing at least linear olefins and branched olefins, is first separated into two streams A and B. Stream A is distilled before the reaction and only the high-boiler phase obtained in this distillation is subjected to the reaction, a heterogeneously catalysed hydroformylation, a homogeneously catalysed hydroformylation or a homogeneously catalysed alkoxycarbonylation.

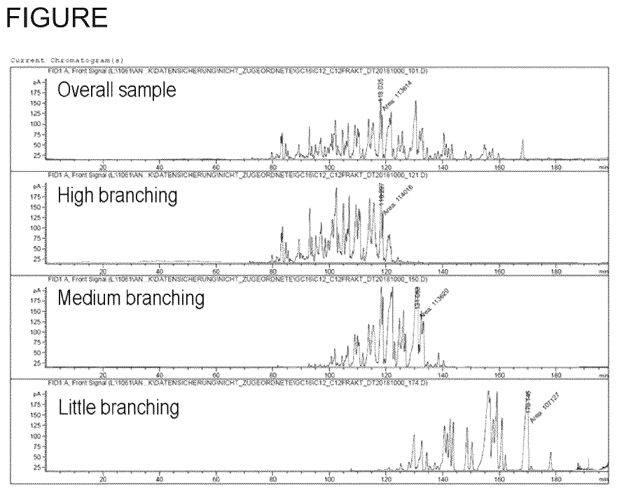

### CROSS-REFERENCE TO RELATED APPLICATIONS

This patent application claims priority to European Patent Application No. 23217518.2, filed on Dec. 18, 2023, in the European Patent Office, the entire disclosure of which is hereby incorporated by reference herein.

### BACKGROUND OF THE INVENTION

### Field of the Invention

The invention provides a process for reacting olefins having 4 to 20 carbon atoms, in which the feed stream used, containing at least linear olefins and branched olefins, is first separated into two streams A and B. Stream A is distilled before the reaction and only the high-boiler phase obtained in this distillation is subjected to the reaction, a heterogeneously catalysed hydroformylation, a homogeneously catalysed hydroformylation or a homogeneously catalysed alkoxycarbonylation.

In the context of the present invention, the term “reaction” is understood to mean the three different reactions heterogeneously catalysed hydroformylation, homogeneously catalysed hydroformylation and homogeneously catalysed alkoxycarbonylation, in which the feed streams used, containing the olefins having 4 to 20 carbon atoms, are reacted. These three reaction types are processes of organic chemistry known per se, but are described in more detail in the context of the present invention.

In the hydroformylation, olefins are reacted with synthesis gas, i.e. a mixture of carbon monoxide (CO) and hydrogen (H₂), in the presence of a suitable homogeneous catalyst system to form the corresponding aldehydes. Hydroformylation is an industrially used process operated in plants in which it is possible to produce the aldehydes on a scale of several to hundreds of kilotons (kt) per year. Catalyst systems typically used here are homogeneously dissolved catalyst systems and comprise transition metal complexes of mostly cobalt or rhodium as metal and phosphorus-containing ligands. Numerous examples of these can be found in the patent literature.

### DESCRIPTION OF RELATED ART

In recent years, more interest has once again been shown in heterogeneously catalysed hydroformylations. EP 3 632 885 A1 discloses one example of corresponding hydroformylations. In said document, instead of the known homogeneously dissolved catalysts, use is made of catalyst systems present in heterogenized form on a monolith support composed of a porous ceramic material. The heterogenized catalyst systems are in particular the transition metal complexes of mostly cobalt or rhodium as metal and phosphorus-containing ligands that are known from the homogeneously catalysed hydroformylation or are comparable thereto.

Alkoxycarbonylation is the reaction of an olefin with carbon monoxide and an alcohol to form the resulting esters. This usually involves the use of metal-ligand complexes as catalysts, which are present homogeneously dissolved in the reaction mixture and thus also in the product mixture. EP 3 750 620 A1 for example discloses a corresponding process. The catalyst systems used comprise in particular a metal from group 8 to 10 of the Periodic Table of the Elements (PTE), for example palladium, or a compound thereof, a phosphorus-containing ligand and an acid as cocatalyst.

Being able to operate the abovementioned reaction processes, i.e. the homogeneously catalysed hydroformylation, the heterogeneously catalysed hydroformylation or the homogeneously catalysed alkoxycarbonylation, in an economically viable manner depends on various factors. One of these factors is the reaction speed, which should be as high as possible. Ultimately, more product can be produced per unit time in comparison with a reaction having a lower reaction speed. However, care should be taken here that other reaction parameters, such as the reaction conversion and/or the selectivity for the desired product, are not impaired in the case of an increase in the reaction speed, so that the advantage of the higher reaction speed is not reversed.

A further problem exists in the case of high mass flow rates of the feed streams. In order to be able to process these, large reaction and distillation plants are required in order to be able to handle high mass flow rates. However, large reaction and distillation plants are accompanied by high capital costs. On the other hand, some reaction and distillation plants can be designed to be sufficiently large to be able to handle high mass flow rates.

### SUMMARY OF THE INVENTION

The object of the present invention was therefore to provide a process for reacting olefins, which makes it possible to achieve a relatively high reaction speed, while the reaction conversion and/or the selectivity for the desired product, here an aldehyde in the case of the hydroformylation or an ester in the case of the alkoxycarbonylation, are not significantly impaired. In addition, high amounts of feed streams should also be able to be processed.

### BRIEF DESCRIPTION OF THE DRAWINGS

The Figure shows an overview of the GC fractions of the tributene streams.

### DETAILED DESCRIPTION OF THE INVENTION

The object was achieved by the process according to the description herein. Preferred configurations are also specified in the dependent embodiments. The process according to the invention is a process for reacting olefins having 4 to 20 carbon atoms, comprising the following steps:
a) providing a feed stream comprising at least linear olefins and branched olefins each having the same number of carbon atoms;
b) dividing the feed stream provided in step a) into at least two streams, a stream A and a stream B;
c) distilling stream A in at least one distillation column to obtain at least one low-boiler phase and one high-boiler phase, where 1% by weight to less than 50% by weight of the distilled feed stream is obtained as low-boiler phase and where the proportion of linear olefins in the low-boiler phase is less than the proportion of the linear olefins in the high-boiler phase; and
d) supplying the high-boiler phase from step c) to a reaction unit comprising one or more reactors, and carrying out a reaction in the reaction unit, where the reaction is either a heterogeneously catalysed hydroformylation in the presence of synthesis gas, a homogeneously catalysed hydroformylation in the presence of synthesis gas or a homogeneously catalysed alkoxycarbonylation in the presence of carbon monoxide and a C1 to C6 alcohol.

One of the crucial process steps is the separation in step b). This involves separating the feed stream used into at least two streams A and B. The separation ensures that a single large feed stream is divided into several smaller streams. This makes it easier to process these smaller streams.

A further crucial process step here is step c), the distillation of the feed stream and the exclusive use of the high-boiler phase for the reaction in step d). The process according to the invention has the advantage that the reaction speed in the reaction in step d) is higher than in the case of the known processes in which there is no distillation of this kind. The reason for this is likely that relatively unreactive isomers and/or reaction-inhibiting substances tend to be separated from the feed stream in the distillation with the low-boiler phase and thus not supplied to the reaction.

The first step a) of the process according to the invention is the provision of a feed stream comprising the olefins having 4 to 20 carbon atoms, preferably olefins having 7 to 16 carbon atoms, particularly preferably olefins having 7 to 12 carbon atoms. Very particular preference is given to feed streams comprising olefins having 8 and/or 12 carbon atoms. The feed streams used are usually industrially available hydrocarbon streams containing at least different isomers of the respective olefin. These feed streams contain at least linear olefins and branched olefins each having the same number of carbon atoms. The amount of the respective olefins in the hydrocarbon streams should understandably be sufficiently high enough to be able to operate the reaction in step d) in an economically viable manner; preferably, the feed streams should contain at least 5% by weight of the olefins in question, based on the total weight of the feed stream.

Feed streams containing olefins having 4 carbon atoms, for example mixtures of the different isomers of butene (1-butene, 2-butene, isobutene), are usually light petroleum fractions from refineries, C4 fractions from FC crackers or steam crackers, mixtures from Fischer-Tropsch syntheses, mixtures from dehydrogenation of butanes and mixtures formed by metathesis or from other industrial processes having partially different concentrations of the isomers. For example, mixtures of linear butenes suitable for the process according to the invention may be obtained from the C4 fraction of a steam cracker. Olefins having 5 carbon atoms, i.e. pentenes, are present in light petroleum fractions from refineries or crackers.

The higher olefins may in particular be obtained by oligomerization reactions, for example dimerization, trimerization or tetramerization. Suitable hydrocarbon streams are also the mixture of isomeric hexenes (dipropene) obtained in the dimerization of propene, the mixture of isomeric octenes (dibutene) obtained in the dimerization of butenes, the mixture of isomeric nonenes (tripropene) obtained in the trimerization of propene, the mixture of isomeric dodecenes (tetrapropene or tributene) obtained in the tetramerization of propene or the trimerization of butenes, the isomeric hexadecene (tetrabutene) obtained in the tetramerization of butenes, and olefin mixtures produced by cooligomerization of olefins having different numbers of carbon atoms (preferably 2 to 4 carbon atoms), optionally after distillative separation into fractions having the same or different numbers of carbon atoms. Olefins or olefin mixtures produced by Fischer-Tropsch synthesis may also be used. It is additionally possible to use olefins produced by olefin metathesis or by other industrial processes.

The feed streams provided in step a) may have been previously subjected to one or more further process steps in order to convert certain components of the feed streams or to remove them from the stream. One example is the removal of impurities that may be harmful to the catalyst, for example substances or compounds containing oxygen, nitrogen or sulfur. One option for removing impurities of this type is to pass the feed stream over an adsorber bed, where the impurities remain attached in the adsorber. Such processes are known and have been published numerous times.

The feed stream provided in step a) is divided in step b) into at least two streams, stream A and stream B. It is also possible in principle to divide the feed stream into more than two streams. The further streams would then be designated with the subsequent capital letters C, D, etc. In this context, “division” means the separation of a total stream into two or more individual streams with identical composition. The division is therefore effected not by means of thermal separation (for example distillation), but for example via distributors in the pipelines. This is known to those skilled in the art. The division may also be adjusted on the basis of a previously determined parameter (mass flow, amount, etc.) and thus optionally automated. Suitable for this purpose would be for example ratio regulators with split range or motor step with position feedback.

Stream A obtained from step b) is subjected in step c) to a distillation, in which a high-boiler phase and a low-boiler phase are obtained. The distillation in step c) is preferably carried out at an overhead temperature in the range from 40° C. to 100° C., particularly preferably in the range from 45° C. to 80° C. The pressure at the top in the distillation in step c) is preferably 50 to 400 mbar, particularly preferably 80 to 350 mbar. The temperature in the bottom in the distillation in step c) is preferably 60° C. to 150° C., particularly preferably 70° C. to 140° C. The pressure in the bottom in the distillation in step c) is preferably 70 to 500 mbar, particularly preferably 90 to 450 mbar. As is known, in the distillation the temperatures present or needed for a specific separation task are dependent on the pressure. Those skilled in the art will be able to select a suitable parameter combination of pressure and temperature on the basis of the separation task to be carried out.

In the distillation, relatively unreactive isomers and/or reaction-inhibiting substances can be at least partially transferred into the low-boiler phase, as a result of which the reaction of the high-boiler phase in step d) can be effected with a relatively high reaction speed. 1% by weight to less than 50% by weight, preferably 5% by weight to 40% by weight, particularly preferably 10% by weight to 30% by weight, of the distilled feed stream is obtained as low-boiler phase in the distillation in step c). The removal of the low-boiler phase in the distillation may be effected here in different ways, which is fundamentally familiar to those skilled in the art. For example, the removal of the specific amount of the low-boiler phase may thus be effected via the separation sharpness of the distillation, which can be influenced on the basis of the reflux ratio and/or the temperature. Also possible would be a removal according to the mass removed, i.e. for example via a mass flow meter or similar suitable apparatuses or internals. The proportion or the amount of feed stream removed as low-boiler phase in the distillation in step c) may alternatively be set on the basis of other parameters.

The result of the distillation in step c) is that the proportion of linear isomers of the olefins used in the low-boiler phase is less than the proportion of the linear isomers of the olefins used in the high-boiler phase. This may be checked or determined for example by means of NMR or gas chromatography. The branched isomers are usually lower-boiling and are transferred into the low-boiler phase in a larger proportion. Since, in the context of the present invention, the olefins used are generally a mixture of isomeric olefins, it is almost impossible to prevent linear isomers from also being transferred into the low-boiler phase. The distillation should however only be carried out in suitable conditions to make it possible for the proportion of the linear isomers to be higher in the high-boiler phase.

The distillation in step c) is carried out in at least one distillation column, i.e. can be carried out in either one or more distillation columns. If only a single distillation column is present, the stream needed for the reaction in step d) is removed from the distillation column as bottom stream. If multiple distillation columns are present, only the high-boiler phase from the first distillation column is supplied to the following distillation column and the low-boiler phase from the last distillation column is supplied to the reaction in step d).

All known distillation columns are suitable in principle for the distillation according to the invention in step c). These distillation columns typically have internals for improving the separation sharpness. Suitable internals are for example trays, unstructured packings (random packings) or structured packings. Trays used are typically bubble-cap trays, sieve trays, valve trays having fixed or movable valves, tunnel-cap trays or slotted trays. Unstructured packings are generally beds of random packings. Random packings used are typically Raschig rings, Pall rings, Berl saddles, Super-Rings/Super-Rings Plus or Intalox® saddles. Structured packings are sold for example under the trade name Mellapak® by Sulzer. In addition to the internals mentioned, further suitable internals are known to those skilled in the art and may likewise be used. In a preferred embodiment, the at least one distillation column in step c) comprises 10 to 100 trays, preferably 15 to 80 trays.

The conditions of the distillation depend on the feed composition and are variable within broad ranges. For very pure streams comprising only minor proportions, if any, of compounds having more or fewer carbon atoms, a single-stage distillation is usually sufficient. The more linear isomers that are to be reacted in step d) are then obtained in the bottoms in the distillation. The more branched isomers that are removed are obtained at the top of the single-stage distillation. If the feed streams used contain significant amounts of compounds having long-chain carbon atoms, a multi-stage distillation may be advantageous, in which the desired stream for step c) is removed as overhead stream from the second or last distillation column.

The high-boiler phase obtained from the distillation in step c) is then supplied in step d) to a reaction in a reaction unit. The reaction unit for the reaction in step d) may consist of one or more reactors. The reactor(s) may in particular be selected from the group consisting of a stirred-tank reactor, a loop reactor, a jet-loop reactor, a bubble-column reactor or combinations thereof. If multiple reactors are present, the reactors may be identical or different. If multiple reactors are present, they may be connected in parallel or in series or be arranged in a hybrid of parallel and serial connection.

If the reaction is a homogeneously catalysed reaction, the following process conditions are preferred:

The olefins used in the process are hydroformylated with synthesis gas in the presence of a homogeneously dissolved catalyst system. The molar ratio between synthesis gas and the feed mixture should be between 6:1 and 1:1, preferably between 3:1 and 1:1, particularly preferably between 2:1 and 1:1. The hydroformylation may optionally be carried out in the presence of a solvent known to those skilled in the art.

The homogeneous catalyst system usable in the hydroformylation may comprise Co or Rh, preferably Rh, and preferably a phosphorus-containing ligand. Phosphorus-containing ligands are practically not absolutely necessary in the case of Co. Appropriate catalyst systems are familiar to those skilled in the art. In a particularly preferred embodiment, the homogeneous catalyst system comprises or consists of Rh and a phosphorus-containing ligand. Suitable ligands for the catalyst systems according to the invention are known to those skilled in the art (see e.g. the textbooks “Rhodium Catalyzed Hydroformylation” (from 2002) by P. W. N. M. van Leeuwen or “Hydroformylation-Fundamentals, Processes and Applications in Organic Synthesis” (from 2016) by A. Börner and R. Franke).

The phosphorus-containing ligand for the catalyst system according to the invention is preferably a phosphine (for example TPP (triphenylphosphine)), a monophosphite (for example Alkanox 240 (tris(2,4-di-tert-butylphenyl)phosphite)) or a bisphosphite (for example BiPhePhos). It is also possible to use mixtures of ligands.

The temperature in the homogeneously catalysed hydroformylation is preferably in the range from 80° C. to 250° C., further preferably in the range from 90° C. to 225° C. and particularly preferably in the range from 100° C. to 210° C. The pressure in the homogeneously catalysed hydroformylation is preferably in the range from 20 to 350 bar, further preferably in the range from 30 to 325 bar and particularly preferably in the range from 45 to 300 bar.

The pressure in the hydroformylation usually corresponds to the total gas pressure. In the context of the present invention, the total gas pressure means the sum of the pressures occurring of all gaseous substances present, i.e. the pressure of the (total) gas phase. In the present process, this corresponds in particular to the sum of the partial pressures of CO and H₂, i.e. the total gas pressure is then the synthesis gas pressure.

Homogeneously catalysed hydroformylations may be operated as liquid discharge processes (“liquid recycle”) or as gas discharge processes (“gas recycle”). Both process variants are known to those skilled in the art and described in numerous textbooks. A specific selection of such a process is not necessary in the context of the present invention because the process can fundamentally be carried out in both ways. What is important in any case in homogeneous catalysis is still the removal of the catalyst system from the reaction output. In the case of a liquid output, this is possible for example via flash processes or membrane separation, and in the case of a gaseous output, for example by means of condensation and/or scrubbing. This too is known to those skilled in the art and does not require a detailed explanation. The further workup of the reaction output, in particular the removal of the reaction product, is likewise familiar to those skilled in the art and may be carried out for example by means of a thermal separation process such as distillation. In the context of the present invention, thermal separation or thermal separation process means a separation process in which the separation is effected on the basis of boiling point.

If the reaction is a heterogeneously catalysed hydroformylation, the following process conditions are preferred:

In the context of the present invention, heterogeneously catalysed hydroformylations are in particular those in which the catalyst system is heterogenized, particularly by immobilization on a supporting material (cf. introductory discussion in WO 2015/028284 A1). The terms heterogenization and immobilization should therefore be understood to mean that the catalyst system is present immobilized by formation of a thin liquid film on the surface and/or in the pores of a solid supporting material.

A particular feature of the heterogeneously catalysed hydroformylation is that the high-boiler phase from step c) is passed in gaseous form over a support composed of a porous ceramic material on which the catalyst system, which comprises a metal from group 8 or 9 of the Periodic Table of the Elements, at least one organic phosphorus-containing ligand and a stabilizer, is present in heterogenized form.

The temperature in the heterogeneously catalysed hydroformylation may be in the range from 65° C. to 200° C., preferably 75° C. to 175° C. and particularly preferably 85° C. to 150° C. The pressure in the heterogeneously catalysed hydroformylation should be greater than 0 bar, but in particular not greater than 35 bar, preferably not greater than 30 bar, particularly preferably not greater than 25 bar. The molar ratio between synthesis gas and the feed mixture should be between 6:1 and 1:1, preferably between 5:1 and 3:1. Optionally, the feed mixture may be diluted with inert gas or a solvent, for example with the alkanes present in technical-grade hydrocarbon streams, in order to control the reaction.

The catalyst system used in the hydroformylation process according to the invention preferably comprises a transition metal from group 8 or 9 of the Periodic Table of the Elements, particularly iron, ruthenium, iridium, cobalt or rhodium, particularly preferably cobalt and rhodium, at least one organic phosphorus-containing ligand, a stabilizer and optionally an ionic liquid.

The stabilizer is preferably an organic amine compound, particularly preferably an organic amine compound containing at least one 2,2,6,6-tetramethylpiperidine unit.

The organic phosphorus-containing ligand for the catalyst system according to the invention preferably has the general formula (I)

$${R}^{′} - A - {R}^{″} - A - {R}^{′′′} \qquad ( I )$$

where R′, R″ and R″ are each organic radicals and each A is a bridging-O—P(—O)₂ group, where two of the three oxygen atoms —O—are respectively bonded to the radical R′ and the radical R″, with the proviso that R′ and R″ are two organic radicals separate from one another. R′ and R″ may be the same or a different organic radical. The organic radicals R′, R″ and R″ preferably do not contain any terminal trialkoxysilane groups.

In a preferred embodiment, R′, R″ and R″ in the compound of formula (I) are preferably selected from substituted or unsubstituted 1,1′-biphenyl, 1,1′-binaphthyl and ortho-phenyl groups, particularly from substituted or unsubstituted 1,1′-biphenyl groups, with the proviso that R′ and R″ are not identical. Particularly preferably, the substituted 1,1′-biphenyl groups have an alkyl group and/or an alkoxy group in the 3,3′ and/or 5,5′ positions of the 1,1′-biphenyl base structure, particularly a C1-C4 alkyl group, particularly preferably a tert-butyl and/or methyl group, and/or preferably a C1-C5 alkoxy group, particularly preferably a methoxy group. One example of a suitable ligand is BiPhePhos (6,6′-[(3,3′-di-tert-butyl-5,5′-dimethoxy-1,1′-biphenyl-2,2′-diyl)bis(oxy)]bis(dibenzo[d,f][1,3,2]dioxaphosphepine)).

The porous ceramic material of which the support is composed is preferably selected from the group consisting of a silicate ceramic, an oxidic ceramic, a nitridic ceramic, a carbidic ceramic, a silicidic ceramic and mixtures thereof. The silicate ceramic is preferably selected from aluminosilicate, magnesium silicate, and mixtures thereof, for example bentonite. The oxidic ceramic is preferably selected from γ-alumina, α-alumina, titanium dioxide, beryllium oxide, zirconium oxide, aluminium titanate, barium titanate, zinc oxide, iron oxides (ferrites) and mixtures thereof. The nitridic ceramic is preferably selected from silicon nitride, boron nitride, aluminium nitride and mixtures thereof. The carbidic ceramic is preferably selected from silicon carbide, boron carbide, tungsten carbide or mixtures thereof. Also conceivable are mixtures of carbidic and nitridic ceramic, what are called carbonitrides. The silicidic ceramic is preferably molybdenum silicide. The support according to the present invention to which the catalyst system is applied preferably consists of a carbidic ceramic.

The support may be a monolith, i.e. the support may consist of a block (a three-dimensional object) of a ceramic material. Such a block may either be in one-piece form or consist of multiple, i.e. at least two, individual parts that may be joined together to form the block and/or may be joined to one another in a fixed or detachable manner. The support may alternatively be in the form of a granular material or in the form of pellets. The median particle diameter (d50) of the support may then be from 0.1 mm to 7 mm, preferably 0.3 to 6 mm, particularly preferably from 0.5 mm to 5 mm. Median particle diameter may be determined by imaging methods, in particular determined by the methods cited in the standards ISO 13322-1 (version: 2004 Dec. 1) and ISO 13322-2 (version: 2006 Nov. 1). The support may be produced in the form of a granular material or in the form of pellets by processes known to those skilled in the art. This may for example be done by mechanically comminuting a monolith of the carbidic material, nitridic material, silicidic material or mixtures thereof, for example using a jaw crusher, and adjusting the particle size of the resulting crushed granular material by sieving.

According to the invention, the support consists of a porous ceramic material, i.e. the ceramic material has pores. Different porosities or pore diameters are conceivable in principle. The pore diameter is however preferably in the range from 0.9 nm to 30 μm, further preferably in the range from 10 nm to 25 μm and particularly preferably in the range from 70 nm to 20 μm. Pore diameter may be determined by nitrogen adsorption or mercury porosimetry in accordance with DIN 66133 (version: 1993-06).

What is called a washcoat, which consists of the same or a different ceramic material with respect to the ceramic material of the support, particularly a ceramic material selected from the abovementioned ceramic materials, preferably silicon oxide, can additionally be applied to the support composed of the ceramic material. The washcoat itself may be porous or nonporous; the washcoat is preferably nonporous. The particle size of the washcoat is preferably 5 nm to 3 μm, preferably 7 nm to 700 nm. The washcoat is used to introduce or to generate the desired pore size and/or to increase the surface area of the support. The washcoat may in particular be applied by dipping (dip-coating) into a washcoat solution containing the ceramic material of the washcoat, optionally also as a precursor. The amount of the washcoat present on the support is ≤20% by weight, preferably ≤15% by weight, particularly preferably ≤10% by weight, based on the total amount of the support. The catalyst system is then applied to the thus produced ceramic support with the washcoat applied. It is however preferred for the support to not comprise a washcoat.

The reaction output obtained in the heterogeneously catalysed hydroformylation does not in principle have to be freed of the catalyst system. The further workup, i.e. for example the removal of the reaction products, is familiar to those skilled in the art and may in principle be carried out by distillation or by another thermal separation process.

If the reaction is a homogeneously catalysed alkoxycarbonylation, the following process conditions are preferred:

In the alkoxycarbonylation, olefins are reacted together with carbon monoxide (CO) and an alcohol, in the present case a C1 to C6 alcohol, in the presence of a homogeneously catalysed catalyst system and esters are formed.

The carbon monoxide may be provided directly as a feed mixture or by adding a carbon monoxide-containing gas selected from synthesis gas, water gas, generator gas and other carbon monoxide-containing gases. It is also possible to provide the carbon monoxide by first separating the carbon monoxide-containing gas into its components in a manner known to those skilled in the art and passing the carbon monoxide to the reaction zone. The carbon monoxide may still contain a certain proportion of hydrogen or other gases, because complete separation is almost impossible to realize.

The alcohol used in the alkoxycarbonylation is a mono- or polyalcohol (polyalcohol=two or more OH groups) having 1 to 6 carbon atoms. Suitable alcohols for the reaction in step d) are methanol, ethanol, 1-propanol, 1-butanol, 1-pentanol, 1-hexanol, 2-propanol, tert-butanol, 3-pentanol, cyclohexanol, phenol or mixtures thereof. Preference is given to using methanol and ethanol in the alkoxycarbonylation. If methanol is used, the reaction is also referred to as a methoxycarbonylation. When using ethanol, the reaction is also referred to as an ethoxycarbonylation.

The homogeneous catalyst system used for the alkoxycarbonylation preferably comprises at least one metal from group 8 to 10 of the Periodic Table of the Elements (PTE) or a compound thereof, a phosphorus-containing ligand and an acid as cocatalyst.

The metal from group 8 to 10 of the PTE is preferably palladium. The palladium is preferably used in the form of a precursor compound as a palladium compound coordinated by the phosphorus-containing ligand. Examples of palladium compounds that may be used as precursor compounds are palladium chloride [PdCl₂], palladium (II) acetylacetonate [Pd(acac)₂], palladium (II) acetate [Pd(OAc)₂], dichloro(1,5-cyclooctadiene) palladium (II) [Pd (cod)₂Cl₂], bis(dibenzylideneacetone) palladium (0) [Pd (dba)₂], tris(dibenzylideneacetone) dipalladium (0) [Pd₂ (dba) 3], bis(acetonitrile)dichloropalladium (II) [Pd(CH₃CN)₂Cl₂], palladium (cinnamyl)dichloride [Pd (cinnamyl) Cl₂]. Preference is given to using the compounds [Pd(acac)₂] or [Pd(OAc)₂]. The concentration of palladium metal in the alkoxycarbonylation is preferably between 0.01 and 0.6 mol %, preferably between 0.03 and 0.3 mol %, particularly preferably between 0.04 and 0.2 mol %, based on the molar amount of the hydrocarbon used.

Suitable phosphorus-containing ligands of the catalyst system according to the invention preferably have a bidentate structure. Preferred phosphorus-containing ligands for the catalyst system according to the invention are benzene-based diphosphine compounds, as disclosed for example in EP 3 121 184 A2. The ligands may be combined with the palladium in a preliminary reaction so that the palladium-ligand complex is passed to the reaction zone, or added to the reaction in situ and combined with the palladium there. The molar ratio of ligand:metal may be 1:1 to 10:1, preferably 2:1 to 6:1, particularly preferably 3:1 to 5:1, in the alkoxycarbonylation.

In the alkoxycarbonylation, the homogeneous catalyst system further comprises an acid, where it may in particular be a Brønsted or a Lewis acid. The Lewis acid used may in particular be aluminium triflate, aluminium chloride, aluminium hydride, trimethylaluminium, tris(pentafluorophenyl) borane, boron trifluoride, boron trichloride or mixtures thereof. Of the Lewis acids mentioned, preference is given to using aluminium triflate. The Lewis acid is preferably added in a molar ratio of Lewis acid:ligand of 1:1 to 20:1, preferably 2:1 to 15:1, particularly preferably 5:1 to 10:1.

Suitable Brønsted acids preferably have an acid strength of pKa≤5, particularly preferably an acid strength of pKa≤3. The stated acid strength pKa relates to the pKa determined under standard conditions (25° C., 1.01325 bar). For a polyprotic acid, the acid strength pKa relates in the context of this invention to the pKa of the first protolysis step. The Brønsted acid is preferably added in a molar ratio of Brønsted acid:ligand of 1:1 to 15:1, preferably 2:1 to 10:1, particularly preferably 3:1 to 5:1.

Brønsted acids used may in particular be perchloric acid, sulfuric acid, phosphoric acid, methylphosphonic acid or sulfonic acids. Suitable sulfonic acids are for example methanesulfonic acid, trifluoromethanesulfonic acid, tert-butanesulfonic acid, p-toluenesulfonic acid (PTSA), 2-hydroxypropane-2-sulfonic acid, 2,4,6-trimethylbenzenesulfonic acid and dodecyl sulfonic acid. Particularly preferred acids are sulfuric acid, methanesulfonic acid, trifluoromethanesulfonic acid and p-toluenesulfonic acid. The acid is preferably sulfuric acid. Carboxylic acids, on the other hand, are less suitable or not suitable at all.

The homogeneously catalysed alkoxycarbonylation is preferably carried out at a temperature of 25° C. to 140° C., further preferably at a temperature of 60° C. to 120° C. and particularly preferably at a temperature of 70° C. to 110° C. The pressure may be between 5 and 60 bar, preferably between 10 and 40 bar, particularly preferably between 15 and 30 bar.

The homogeneously catalysed alkoxycarbonylation affords a product mixture comprising at least the ester formed by the reaction, the homogeneous catalyst system, low boilers, for example low-boiling by-products such as ethers, high boilers, unreacted alcohols and any unreacted hydrocarbons. The product mixture may therefore be subjected to a subsequent catalyst removal. This may for example be effected with a membrane separation, whereby the homogeneous catalyst system and unreacted hydrocarbon and/or unreacted alcohol are enriched in the retentate, while the ester formed is enriched in the permeate. The retentate comprising the enriched homogeneous catalyst system may then be recycled into the reaction zone.

The further workup of the permeate, in particular the removal of the esters as target products, may be effected by known processes and is familiar in principle to those skilled in the art. Thermal separation processes such as distillation are one option.

Stream B obtained in the separation in step b) and any further streams obtained in the separation may likewise be subjected to a distillation before a reaction, in order to remove low-boiling or highly branched isomers. The conditions here may be the same as those presented above for stream A. It will be apparent that the distillation is then carried out in its own distillation unit and not in the same distillation unit in which stream A is distilled. It would also be possible in principle for stream B to be separated once again and for only at least part to be supplied to a subsequent process step.

It is preferred according to the invention that at least part of stream B is passed to a reaction unit comprising one or more reactors and is subjected to a reaction in the reaction unit, where the reaction is either a heterogeneously catalysed hydroformylation in the presence of synthesis gas, a homogeneously catalysed hydroformylation in the presence of synthesis gas, a homogeneously catalysed alkoxycarbonylation in the presence of carbon monoxide and a C1 to C6 alcohol or is a hydrogenation.

The heterogeneously catalysed hydroformylation in the presence of synthesis gas, the homogeneously catalysed hydroformylation in the presence of synthesis gas and the homogeneously catalysed alkoxycarbonylation in the presence of carbon monoxide and a C1 to C6 alcohol may have the same features, as have already been described previously for the reaction of the high-boiler phase in step d).

If the reaction to which at least part of stream B is subjected is a hydrogenation, olefins present are hydrogenated to form the corresponding alkanes. The hydrogenation is effected in a hydrogenation unit, which may consist of one or more reactors. The reactors may be operated in a straight pass or in circulation mode. In a preferred embodiment of the present invention, the hydrogenation unit comprises at least two reactors. Preferably, the first reactor is operated in circulation and the second reactor is operated in a straight pass. The first and the second reactor here may be connected to one another via an overflow. This has the advantage that a pump does not have to be used between the first and the second reactor. Hydrogen is used in the hydrogenation preferably in a stoichiometric excess, particularly preferably in a stoichiometric excess of 5% to 30%.

The hydrogenation of the at least one part of stream B may be carried out on suitable and known supported catalysts. Suitable supported catalysts comprise at least one transition metal from the group consisting of palladium, platinum, rhodium, ruthenium, nickel or mixtures thereof and a supporting material from the group consisting of aluminium oxide, silicon dioxide, titanium dioxide, magnesium oxide or mixtures thereof. In a preferred embodiment of the present invention, use is made in the hydrogenation in the optional step of a supported catalyst which contains palladium or nickel as transition metal.

The hydrogenation is preferably carried out at a temperature of 100° C. to 180° C., particularly preferably at a temperature of 135° C. to 160° C. The pressure is preferably 5 to 40 barg, particularly preferably 10 to barg, in the hydrogenation. The pressure is generated here in particular by the gas phase, i.e. the hydrogen. The hydrogenation is preferably carried out in the liquid phase. A phase separation known to those skilled in the art may be carried out after the hydrogenation in order to separate the gas phase, which comprises unconverted hydrogen and possibly also small amounts of hydrocarbons, from the liquid phase.

It has already been mentioned that the separation in step b) is regulated. One possible parameter that can be used to control the separation is the demand for the different reaction products on the market. If for example there is more demand for alkanes or alkane mixtures, the separation in step b) may be controlled such that a greater proportion of the feed stream is passed to the hydrogenation. If, in contrast, there is greater demand for the products of the hydroformylation or the alkoxycarbonylation, the separation in step b) may be controlled such that a greater proportion of the feed stream is passed to these reactions. It is thus preferred that the separation in step b) is varied in such a way that the proportion of the feed stream obtained as stream A changes during the process.

In a preferred embodiment of the present invention, the low-boiler phase obtained in the distillation in step c) may be subjected to a hydrogenation. The olefins present in the low-boiler phase are hydrogenated here to form the corresponding alkanes. The hydrogenation is effected in a hydrogenation unit, which may consist of one or more reactors. The reactors may be operated in a straight pass or in circulation mode. In a preferred embodiment of the present invention, the hydrogenation unit comprises at least two reactors. Preferably, the first reactor is operated in circulation and the second reactor is operated in a straight pass. The first and the second reactor here may be connected to one another via an overflow. This has the advantage that a pump does not have to be used between the first and the second reactor. Hydrogen is used in the hydrogenation preferably in a stoichiometric excess, particularly preferably in a stoichiometric excess of 5% to 30%.

The hydrogenation of the low-boiler phase may be carried out on suitable and known supported catalysts. Suitable supported catalysts comprise at least one transition metal from the group consisting of palladium, platinum, rhodium, ruthenium, nickel or mixtures thereof and a supporting material from the group consisting of aluminium oxide, silicon dioxide, titanium dioxide, magnesium oxide or mixtures thereof. In a preferred embodiment of the present invention, use is made in the hydrogenation in the optional step of a supported catalyst which contains palladium or nickel as transition metal.

The hydrogenation is preferably carried out at a temperature of 100° C. to 180° C., particularly preferably at a temperature of 135° C. to 160° C. The pressure is preferably 5 to 40 barg, particularly preferably 10 to barg, in the hydrogenation. The pressure is generated here in particular by the gas phase, i.e. the hydrogen. The hydrogenation is preferably carried out in the liquid phase. A phase separation known to those skilled in the art may be carried out after the hydrogenation in order to separate the gas phase, which comprises unconverted hydrogen and possibly also small amounts of hydrocarbons, from the liquid phase.

### Examples

Hydroformylation of Tributene 49 kg of tributene (feed) was distilled in a distillation column (80 I) having several packing beds (Montz A3-1000) at a temperature of 70° C. (top) or 110° C. (bottom) and a pressure of 90 mbar (top) or 120 mbar (bottom). 20% to 25% was removed as distillate (=low boilers) from the tributene used. The remaining 75% to 80% remain in the bottoms and are therefore high boilers.

The three different tributene streams feed, distillate and bottoms were investigated with respect to their composition. Since the identification of the individual isomers of tributene is difficult on account of the high number of isomers, the tributene streams were investigated with respect to the proportion of different fractions by means of gas chromatography (GC capillary column, Petrocol, DH 150). For simplification, the following were therefore identified on the basis of their retention times:
isomers with high branching having retention times of 70 to 109 min,
isomers with medium branching having retention times of 109 to 134 min, and
isomers with little branching having retention times of 134 to 180 min

An overview of the proportion of the respective abovementioned fractions in the tributene streams can be seen in Table 1.

TABLE 1
Proportion of the fractions in the respective
tributene streams (mass %)

| | Feed (before | Distillate (low | Bottoms (high |
|---|---|---|---|
| | distillation) | boilers) | boilers) |
| Isomers with high | 23.5% | 93.5% | 0% |
| branching | | | |
| Isomers with | 61% | 6.5% | 79% |
| medium branching | | | |
| Isomers with little | 15.5% | 0% | 21% |
| branching | | | |

It is apparent from the analysis of the feed, distillate and bottoms that the distillation causes primarily isomers of the tributene with high branching to be removed from the feed and end up in the distillate. The proportion of isomers with little branching is significantly higher in the bottoms than in the feed or in the distillate.

A hydroformylation was carried out in each case with the three tributene streams feed, distillate and bottoms in 100 ml autoclaves. The catalyst used here was rhodium (40 ppm of Rh) with a ligand (Alkanox 240) in a 5-fold molar excess (ratio of overall phosphorus to rhodium). The temperature was 130° C. to 150° C. Furthermore, the hydroformylations were carried out in each case at a synthesis gas pressure (CO/H₂ ratio=1:1 (vol %)) of 235 to 250 bar. The solvent used was 40 to 46 g of toluene in each case. Samples were taken in each case at 10 minutes, 60 minutes and 180 minutes after the start of the reaction and investigated with respect to the reaction conversion (conversion=molar amount at time t/molar amount (beginning of the reaction)). The results of the hydroformylation are shown in Table 2.

TABLE 2
Reaction conversion in the hydroformylation

| | Conversion/% | | |
|---|---|---|---|
| | After 10 | After 60 | After 180 |
| | minutes | minutes | minutes |
| Feed (before | 38.8 | 80.4 | 90.9 |
| distillation) | | | |
| Distillate (low boilers) | 5.9 | 32.51 | 57.8 |
| Bottoms (high boilers) | 43.3 | 85.12 | 94.2 |

It can be inferred from Table 2 that a significant acceleration of the reaction can be achieved by way of a prior distillation. Compared with the feed, the conversion when using the bottom stream is in each case about 5% higher.

### Hydroformylation of Dibutene

21.5 t/h of dibutene (feed) was distilled in a distillation column having 42 trays at an overhead temperature of 58° C. and a bottom temperature of 62° C. and corresponding overhead pressure of 0.1 bar and a bottom pressure of 0.15 bar. About 20% was removed (4.44 t/h) as distillate (=low boilers) from the dibutene used. The remaining 80% was taken off in the bottoms and is therefore high boilers.

The dibutene streams feed and bottoms were investigated with respect to their composition by means of GC (gas chromatography). An overview is shown in Table 3:

TABLE 3
Proportion of different groups of isomers
in the respective dibutene stream

| | Feed (before | Bottoms (high |
|---|---|---|
| | distillation) | boilers) |
| Dimethylhexenes | 19% | 8% |
| Methylheptenes | 65% | 72% |
| Octenes | 16% | 20% |

The linearity of dibutene is described by the ISO index and represents a value for the average number of methyl branches in the dimer. For example (for butene as the reactant), n-octenes thus contribute 0, methylheptenes contribute 1 and dimethylhexenes contribute 2 to the ISO index of a C8 fraction. The lower the ISO index, the more linear the structure of the molecules in the respective fraction. The ISO index is calculated according to the following general formula, where the proportion of the individual dimer fractions is based on the total dimer fraction:

$$ISO index = \frac{\begin{matrix}( singly branched dimers ( \% by weight ) + \\ 2 \times doubly branched dimers ( \% by weight ) )\end{matrix}}{100}$$

Accordingly, a dimer mixture having an ISO index of 1.0 has an average of precisely one methyl branch per dimeric molecule.

The ISO index of the feed is 1.03 and the ISO index of the bottoms is 0.88. It is apparent that the bottoms contains a higher proportion of linear isomers.

A hydroformylation was carried out in each case with the feed and the bottoms in 100 ml autoclaves. The catalyst used here was rhodium (20 ppm of Rh) with a ligand (tris(2,4-di-tert-butylphenyl)phosphite (TDTBPP)) in a 5-fold molar excess (ratio of overall phosphorus to rhodium). The temperature was about 140° C. Furthermore, the hydroformylations were carried out in each case at a synthesis gas pressure (CO/H₂ ratio=1:1 (vol %)) of about 235 bar. The solvent used was 150 ml of toluene in each case. Samples were taken in each case at 10 minutes, 30 minutes, 60 minutes and 180 minutes after the start of the reaction and investigated with respect to the reaction conversion (conversion=molar amount at time t/molar amount (beginning of the reaction)). The results of the hydroformylation are shown in Table 4.

TABLE 4
Reaction conversion in the hydroformylation
of the dibutene streams

Conversion/%

| | After 10 | After 30 | After 60 | After 180 |
|---|---|---|---|---|
| | minutes | minutes | minutes | minutes |
| Feed (before | 51 | 79.9 | 90 | 92.5 |
| distillation) | | | | |
| Bottoms (high | 71 | 89.4 | 94 | 95.1 |
| boilers) | | | | |

It can be inferred from Table 4 that a significant acceleration of the reaction can be achieved by way of a prior distillation. Compared with the feed, the conversion when using the bottom stream is in each case up to 20% higher.

1. A process for reacting olefins having 4 to 20 carbon atoms, comprising:
a) providing a feed stream comprising at least linear olefins and branched olefins each having a same number of carbon atoms;
b) dividing the feed stream provided in a) into at least two streams, a stream A and a stream B;
c) distilling stream A in at least one distillation column to obtain at least one low-boiler phase and one high-boiler phase, where 1% by weight to less than 50% by weight of the distilled stream A is obtained as at least one low-boiler phase and where a proportion of linear olefins in the at least one low-boiler phase is less than a proportion of the linear olefins in the high-boiler phase; and
d) supplying the high-boiler phase from c) to a reaction unit comprising one or more reactors, and carrying out a reaction in the reaction unit, where the reaction is either a heterogeneously catalysed hydroformylation in presence of synthesis gas, a homogeneously catalysed hydroformylation in presence of synthesis gas or a homogeneously catalysed alkoxycarbonylation in presence of carbon monoxide and a C1 to C6 alcohol.

2. The process according to claim 1, further comprising:
passing at least part of stream B to a reaction unit comprising one or more reactors and subjecting at least part of stream B to a reaction in the reaction unit, wherein the reaction is either a heterogeneously catalysed hydroformylation in presence of synthesis gas, a homogeneously catalysed hydroformylation in presence of synthesis gas or a homogeneously catalysed alkoxycarbonylation in presence of carbon monoxide and a C1 to C6 alcohol or is a hydrogenation.

3. The process according to claim 1, further comprising:
reacting olefins having 7 to 16 carbon atoms in the process.

4. The process according to claim 1, wherein a homogeneous catalyst system in the homogeneously catalysed hydroformylation comprises Co or Rh.

5. The process according to claim 1, wherein a homogeneous catalyst system in the homogeneously catalysed alkoxycarbonylation comprises a metal from group 8 to 10 of the Periodic Table of the Elements (PTE) or a compound thereof, a phosphorus-containing ligand and an acid as cocatalyst.

6. The process according to claim 1, further comprising:
subjecting the at least one low-boiler phase obtained in c) to a hydrogenation.

7. The process according to claim 6, further comprising:
carrying out the hydrogenation at a temperature of 100° C. to 180° C.

8. The process according to claim 6, further comprising:
using hydrogen in stoichiometric excess in the hydrogenation.

9. The process according to claim 6, further comprising:
using a supported catalyst comprising at least one transition metal from the group consisting of palladium, platinum, rhodium, ruthenium, nickel or mixtures thereof and a supporting material from the group consisting of aluminium oxide, silicon dioxide, titanium dioxide, magnesium oxide and mixtures thereof in the hydrogenation.

10. The process according to claim 1, further comprising:
carrying out the distillation in c) at an overhead temperature in a range from 40° C. to 100° C.

11. The process according to claim 1, wherein a pressure at a top in the at least one distillation column in b) is 50 to 250 mbar.

12. The process according to claim 1, wherein a temperature in a bottom in the at least one distillation column in c) is 60° C. to 150° C.

13. The process according to claim 1, further comprising:
varying the separation in b) in such a way that a proportion of the feed stream obtained as stream A changes during the process.

14. The process according to claim 1, wherein the at least one distillation column comprises internals, wherein internals are selected from the group consisting of trays, unstructured and/or random packings and structured packings.

15. The process according to claim 1, further comprising:
obtaining 5% by weight to 40% by weight of the distilled feed stream as at least one low-boiler phase in c).
